(19) United States
(12) Patent Application Publication
(10) Pub. No.: US 2011/0306869 A1
(43) Pub. Date: Dec. 15, 2011

(54) APPARATUS AND METHOD FOR PRODUCING TOMOGRAPHIC IMAGE

(75) Inventors: Do-Sik Hwang, Seoul (KR); Ji-Han Kim, Seoul (KR); Young-Jin Kim, Seoul (KR)

(73) Assignee: Industry-Academic Cooperation Foundation, Yonsei University, Seoul (KR)

(21) Appl. No.: 13/160,777

(22) Filed: Jun. 15, 2011

(30) Foreign Application Priority Data
Jun. 15, 2010 (KR) 10-2010-0056534
Sep. 20, 2010 (KR) 10-2010-0092543
Apr. 27, 2011 (KR) 10-2011-0039531

(51) Int. Cl. A61B 8/14; A61B 5/055

(52) U.S. Cl. 600/411; 600/443

(57) ABSTRACT

An apparatus and method for producing tomographic image is disclosed. The disclosed apparatus for producing tomographic image comprises: an ultrasonic imaging unit configured to obtain multiple ultrasonic images of a subject; a tomographic imaging unit configured to obtain one or more tomographic image data of the subject; and a tomographic image correction unit configured to produce corrected tomographic image of a tomographic image which can be produced from the one or more tomographic image data by using the multiple ultrasonic images.

### CROSS-REFERENCE TO RELATED APPLICATION

This application claims under 35 U.S.C. §119(a) the benefit of Korean Application Nos. 10-2010-0056534 filed on Jun. 15, 2010, 10-2010-0092543 filed on Sep. 20, 2010, and 10-2011-0039531 filed on Apr. 27, 2011, the entire contents of which applications are incorporated herein by reference.

### TECHNICAL FIELD

The present invention relates to an apparatus and method for producing tomographic image, more particularly to an apparatus and method for producing tomographic image that can obtain accurate tomographic image with motion correction.

### BACKGROUND ART

Due to such reasons as development in medical science and decrease in birth rate, the average age of populations in developed countries such as the United States, Japan, and Europe is becoming higher. Accordingly, demand for healthcare industry catering to the elderly is also increasing.

Especially, the elderly suffer from such illnesses as cerebral diseases, cardiovascular diseases, and cancers, and accordingly, there is an increasing interest in image diagnostic equipment for accurate diagnosis of the aforementioned illnesses, such as magnetic resonance imaging (MRI), computed tomography (CT), positron emission tomography (PET), and single photon emission computed tomography (SPECT).

But, the aforementioned conventional image diagnostic equipment had the problem of having difficulty producing a clear diagnostic image because of the long time required for producing a diagnostic image. In other words, when using the aforementioned conventional diagnostic imaging equipment to produce a diagnostic image, there was the problem of the increasing possibility of the diagnostic image becoming blurry or having a distortion due to the breathing, heartbeat, movement, etc. of the patient during the long time it took to produce the image.

### SUMMARY OF THE DISCLOSURE

The purpose of the present invention is to provide an apparatus and method for producing tomographic image that can obtain accurate tomographic image with motion correction.

Also, another purpose of the present invention is to provide an apparatus and method for producing tomographic image that can indicate tomographic image as a moving image.

Also, another purpose of the present invention is to provide an apparatus and method for producing tomographic image that can obtain accurate tomographic image in real time.

To resolve the problems of the related art addressed above, an embodiment of the invention provides an apparatus for producing tomographic image comprising: an ultrasonic imaging unit configured to obtain multiple ultrasonic images of a subject; a tomographic imaging unit configured to obtain one or more tomographic image data of the subject; and a tomographic image correction unit configured to produce corrected tomographic image of a tomographic image which can be produced from the one or more tomographic image data by using the multiple ultrasonic images.

Also, an embodiment of the invention provides a method for producing tomographic image, comprising: obtaining multiple ultrasonic images of a subject; obtaining one or more tomographic image data of the subject; and producing corrected tomographic image of a tomographic image which can be produced from the one or more tomographic image data by using the multiple ultrasonic images.

The apparatus and method for producing tomographic image according to the present invention has the advantage of being able to obtain accurate tomographic image with motion correction.

Also, the apparatus and method for producing tomographic image according to the present invention has the advantage of being able to display tomographic image as a moving image.

Also, apparatus and method for producing tomographic image according to the present invention has the advantage of being to obtain accurate tomographic image in real time.

### BRIEF DESCRIPTION OF DRAWINGS

FIG. 1 is a drawing for the purpose of explaining the concept of an apparatus for producing tomographic image according to an embodiment of the present invention.

FIG. 2 is a block diagram illustrating a detailed structure of a tomographic image producing apparatus according to an embodiment of the present invention.

FIG. 3 is a drawing for the purpose of explaining an example of a tomographic correction unit producing tomographic image with motion correction, according to an embodiment of the present invention.

FIG. 4 is a drawing for the purpose of explaining an example of a tomographic correction unit producing corrected tomographic image in real time, according to an embodiment of the present invention.

FIG. 5 is a drawing for the purpose of explaining an example of a tomographic correction unit producing corrected tomographic moving images according to an embodiment of the present invention.

FIG. 6 is a drawing illustrating an example of medical equipment using tomographic image according to an embodiment of the present invention.

FIG. 7 is a flow chart illustrating the overall flow of the method of producing tomographic image according to an embodiment of the present invention.

### DETAILED DESCRIPTION OF THE DISCLOSURE

Embodiments of the invention will be described below in more detail with reference to the accompanying drawings.

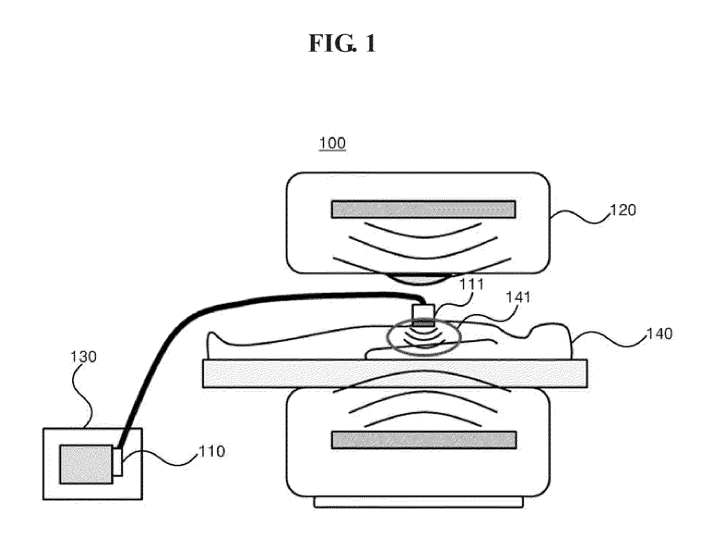

FIG. 1 is a drawing for the purpose of explaining the concept of an apparatus for producing tomographic image according to an embodiment of the present invention.

An apparatus for producing tomographic image 100 according to an embodiment of the present invention is able to obtain ultrasonic images and tomographic image data, and to produce tomographic image by correcting and reconstructing tomographic image data with the use of ultrasonic images. Here, ultrasonic images and tomographic image data should preferably be obtained simultaneously.

Accordingly, an ultrasonic imaging apparatus 110 and a tomographic imaging apparatus 120 are placed as illustrated in FIG. 1, and able to obtain ultrasonic images and the tomographic imaging data simultaneously.

In addition, according to an embodiment of the present invention, since the ultrasonic imaging apparatus 110 and the tomographic imaging apparatus 120 can be operated simultaneously, a shielding frame 130 may be placed on the exterior of the ultrasonic imaging apparatus 110 in order to eliminate the influence of the tomographic imaging apparatus 120 received by the ultrasonic imaging apparatus 110 and vice-versa.

And, according to an embodiment of the present invention, an ultrasonic probe 111 may be used to obtain ultrasonic images, so that ultrasonic images and tomographic image data may be obtained for the same subject area 141.

Meanwhile, according to another embodiment of the present invention, ultrasonic probe 111 is placed within the tomographic imaging apparatus 120.

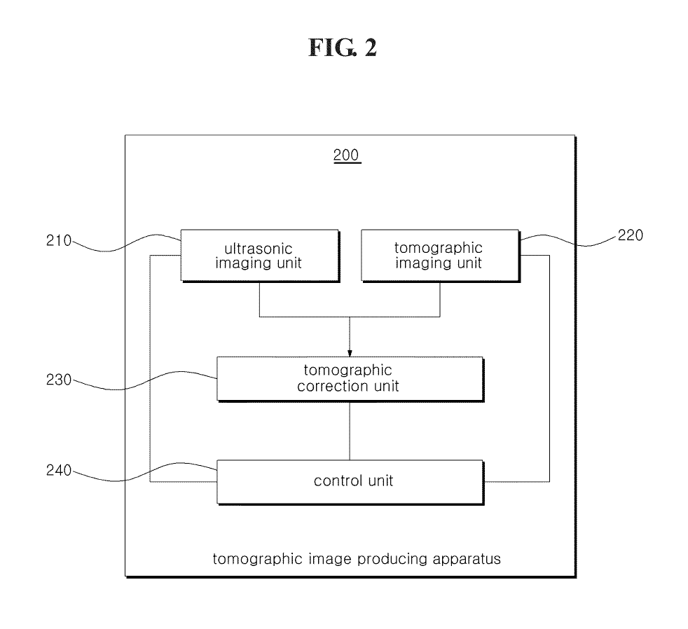

FIG. 2 is a block diagram illustrating a detailed structure of a tomographic image producing apparatus according to an embodiment of the present invention.

Referring to FIG. 2, the apparatus for producing tomographic image 200 according to an embodiment of the present invention may include an ultrasonic imaging unit 210, a tomographic imaging unit 220, a tomographic correction unit 230, and a control unit 240.

The ultrasonic imaging unit 210 produces multiple ultrasonic images for a subject. This may correspond to the ultrasonic imaging apparatus 110 described in FIG. 1.

According to an embodiment of the present invention, the ultrasonic imaging unit 210 can produce multiple ultrasonic images of a subject successively within a pre-set time period.

Also, although not illustrated in FIG. 2, the ultrasonic imaging unit 210 may include an ultrasonic probe (corresponding to the ultrasonic probe 111 described in FIG. 1), which radiates ultrasonic waves, and an ultrasonic probe support which, adjusts the position of the ultrasonic probe and supports the ultrasonic probe. The ultrasonic probe support (not pictured) allows the ultrasonic probe to adhere closely to the subject area, and supports the ultrasonic probe so that the imaging position of tomographic image data by the tomographic imaging unit 220 described below and the imaging position of ultrasonic images by the ultrasonic probe are the same.

The tomographic imaging unit 220 obtains one or more tomographic image data of a subject. This may correspond to the tomographic imaging apparatus 120 described in FIG. 1 above.

According to an embodiment of the present invention, the tomographic imaging unit 220 can produce one or more tomographic image data of a subject successively during the pre-set time period.

Also, according to an embodiment of the present invention, the tomographic imaging unit 220 may include at least one of the following: magnetic resonance imaging (MRI) equipment, CT (computed tomographic image) equipment, positron emission tomographic image (PET) equipment, and single photon emission computed tomographic image (SPECT) equipment. Henceforth, for the sake of ease of explanation, embodiments of the present invention will be described with reference to examples wherein the tomographic imaging unit 220 includes MRI equipment.

Such tomographic imaging unit 220 can divide frequency space into multiple area (frequency area), obtain partial image data from the multiple frequency area for overall tomographic image, and obtain tomographic image data according to time periods by transforming the obtained partial image data into spatial domain. For obtaining tomographic image data for a subject area in frequency space, radial pulse sequence imaging method and spiral pulse sequence imaging method can be used. And besides, various pulse sequence imaging method and be used for obtaining tomographic image data.

From among these, Radial pulse sequence imaging method is a method of obtaining tomographic image data, which can obtain both low-frequency image data and high-frequency image data in frequency space of MRI equipment. Such radial pulse sequence imaging method has the advantage of having data obtained in frequency space corresponding exactly to actual projection images, according to the Fourier-Slice theorem. This is similar to imaging methods used in other types of tomographic imaging equipment such as CT, PET and SPECT equipment.

Such ultrasonic imaging unit 210 and tomographic imaging unit 220 should preferably operate synchronously. This is because the effect of the present invention may be maximized if ultrasonic images and tomographic image data are obtained from the same subject area during the same period of time.

Also, as ultrasonic images are obtained more quickly than tomographic images, the ultrasonic imaging unit 210 can obtain multiple ultrasonic images during the time that the tomographic imaging unit 220 obtains one tomographic image datum. In such case, the multiple ultrasonic images may be used to correct the one tomographic image datum obtained during the same period of time.

At the same time, one or more of the tomographic image data obtained at the tomographic imaging unit 220 may be reconstructed into tomographic image. Here, the reconstructed tomographic image is tomographic image produced by the use of an ordinary conventional tomographic imaging apparatus.

The tomographic correction unit 230 corrects tomographic image which can be produced from one or more of the tomographic image data described above by using the multiple ultrasonic images. In other words, the tomographic correction unit 230 produces corrected images of tomographic image (corrected tomographic image) produced from one or more of the tomographic image data by using the multiple ultrasonic images.

The control unit 240 controls the operation of the ultrasonic imaging unit 210, the tomographic imaging unit 220, and the tomographic correction unit 230.

As an example, the control unit 240 may include a processor for controlling the ultrasonic imaging unit 210, the tomographic imaging unit 220, and the tomographic correction unit 230, and may further include memory for storing specific controlling information.

Also, although not illustrated in FIG. 1, the apparatus for producing tomographic image may further include a display unit for displaying the corrected tomographic image.

Below, operation of a tomographic correction unit 230 according to an embodiment of the present invention will be described in more detail.

According to an embodiment of the present invention, the tomographic correction unit 230 can spatially match tomographic image reconstructed from one or more tomographic image data and multiple ultrasonic images. Here, spatial matching refers to the work of making ultrasonic images and tomographic images to be geometrically aligned (preferably overlayed on the same scene).

As an example, the tomographic correction unit 230 can reconstruct one or more tomographic image data into tomographic image by means of mapping or of filtered back projection in image space.

In other words, radial pulse sequence tomographic image data may be obtained by means of radial pulse sequence imaging of a subject area with the use of MRI equipment, the obtained radial pulse sequence tomographic image data may be given filtered back projection into image space, and thus a tomographic image may be produced.

And, the user may insert or attach an marker to the subject area and obtain ultrasonic images and magnetic resonance tomographic image of the subject area simultaneously, in which case the tomographic correction unit 230 can spatially match the ultrasonic images and the magnetic resonance tomographic image with reference to the marker shown in the ultrasonic images and the magnetic resonance tomographic image.

Next, the tomographic correction unit 230 selects a reference ultrasonic image out of the multiple ultrasonic images, and extracts motion information between the reference ultrasonic image and the multiple ultrasonic images.

According to an embodiment of the present invention, the tomographic correction unit 230 can extract motion information by means of at least one of the following: block matching, feature-based tracking, speckle tracking, and deformation-based motion tracking.

Afterwards, the tomographic correction unit 230 corrects one or more tomographic image data by means of the extracted motion information.

According to an embodiment of the present invention, the tomographic correction unit 230 produces correction information for compensating the motion information corresponding to each of the multiple ultrasonic images, and after adjusting the produced correction information so that it spatially matches with a tomographic image datum, can produce one or more corrected tomographic image data by applying the correction information to each corresponding tomographic image data.

Here, the correction information refers to information produced by re-sorting and refining the motion information with reference to the reference ultrasonic image.

Also, the motion information and correction information for ultrasonic images may be expressed as motion vectors. In other words, the motion information from each ultrasonic image may be expressed as a motion vector, and accordingly the correction information for each ultrasonic image may be expressed as a difference in the motion vector.

Lastly, the tomographic correction unit 230 produces tomographic image with motion corrected by reconstructing one or more tomographic image data to which correction information has been applied (that is, corrected tomographic image data).

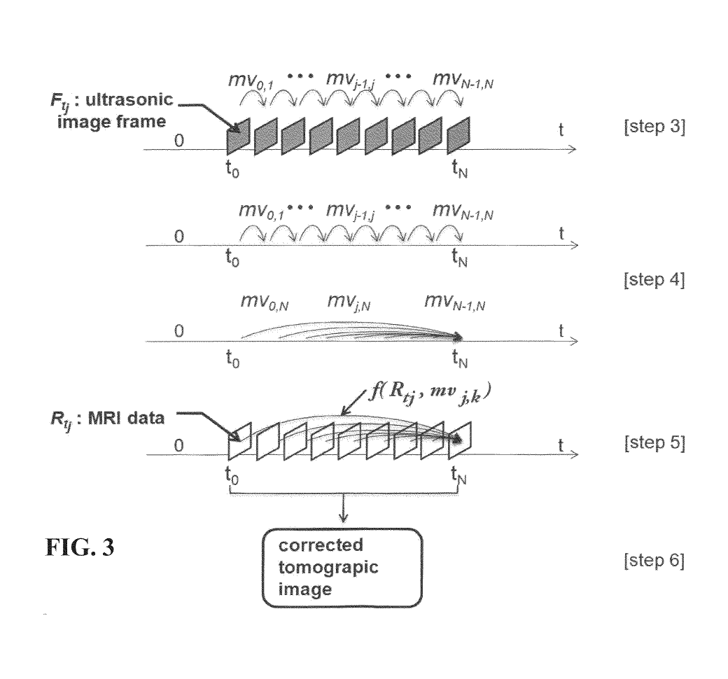

FIG. 3 is a drawing for the purpose of explaining an example of a tomographic correction unit 230 producing tomographic image with motion corrected, according to an embodiment of the present invention. Referring to FIG. 3, sequential explanation of an example of operation of the tomographic correction unit 230 producing corrected tomographic image is as follows.

Step 1) The tomographic correction unit 230 receives N+1 number of ultrasonic images and N+1 number of tomographic image data, obtained between Frames t₀ and t_{N}, from the ultrasonic imaging unit 210 and the tomographic imaging unit 220.

Step 2) The tomographic correction unit 230 selects one ultrasonic image out of the received N+1 number of ultrasonic images as a reference ultrasonic image. As an example, the tomographic correction unit 230 selects an ultrasonic image of Frame t_{N} as the reference ultrasonic image.

Step 3) The tomographic correction unit 230 extracts motion information (N number of pieces of motion information) between the ultrasonic image of Frame t_{N} (that is, the reference ultrasonic image) and the other N number of ultrasonic images.

Step 4) The tomographic correction unit 230 produces N number of pieces of correction information corresponding to each of the N number of ultrasonic images. Here, the N number of pieces of correction information is information for compensating the N number of pieces of motion information.

Step 5) The tomographic correction unit 230, after adjusting the N number of pieces of correction information to match temporally with the N number of tomographic image data excluding the tomographic image data (corresponding to the reference ultrasonic image) obtained from Frame t_{N}, applies the N number of pieces of correction information to each of the N number of tomographic image data.

Step 6) The tomographic correction unit 230 produces corrected tomographic image corresponding to the reference ultrasonic image, combining the N number of corrected tomographic image data to which correction information was applied and the tomographic image data of Frame t_{N}.

And, according to an embodiment of the present invention, the ultrasonic imaging unit 210 can obtain two or more ultrasonic images of a subject area successively, and the tomographic imaging unit 220 can obtain two or more tomographic image data of the same subject area successively; in this case, the multiple ultrasonic images used to produce corrected tomographic image may be the ultrasonic images included within the window size range pre-set with the present time as reference, out of the two or more ultrasonic images. Likewise, the one or more tomographic image data used to produce corrected tomographic image also may be the tomographic image data included within the window size range with the present time as reference, out of the two or more tomographic image data.

Here, the window size range refers to the minimum number of tomographic image data obtained successively for producing one tomographic image.

And, accordingly to an embodiment of the present invention, the reference ultrasonic image selected for producing corrected tomographic image may be the most recently obtained ultrasonic image, with the present time being the reference point, out of the multiple ultrasonic images included within the window size range.

When using the concept of window size range in this manner, since the multiple ultrasonic images and one or more tomographic image data change as time progresses, the tomographic correction unit 230 is able to produce corrected tomographic image in real time.

As an example, the tomographic correction unit 230 produces the corrected tomographic image as illustrated in FIG. 3, since N+1 number of tomographic image data can configure one tomographic image, the window size range is “N+1”, and the tomographic correction unit 230 can select an ultrasonic image obtained from Frame t_{N}, most recently with the present time as reference, as a reference ultrasonic image, out of the N+1 number of ultrasonic images. Also, the motion information (N number of pieces of motion information) extracted between the ultrasonic image of Frame t_{N} (that is, the reference ultrasonic image) and the other N number of ultrasonic images may be expressed as in Formula 1 below.

$${mv}_{j , N} = {\sum }_{k = j}^{N - 1} {mv}_{k , k + 1} \qquad [ Formula 1 ]$$

Here, mv_{j,N} refers to a motion vector indicating motion from j to N.

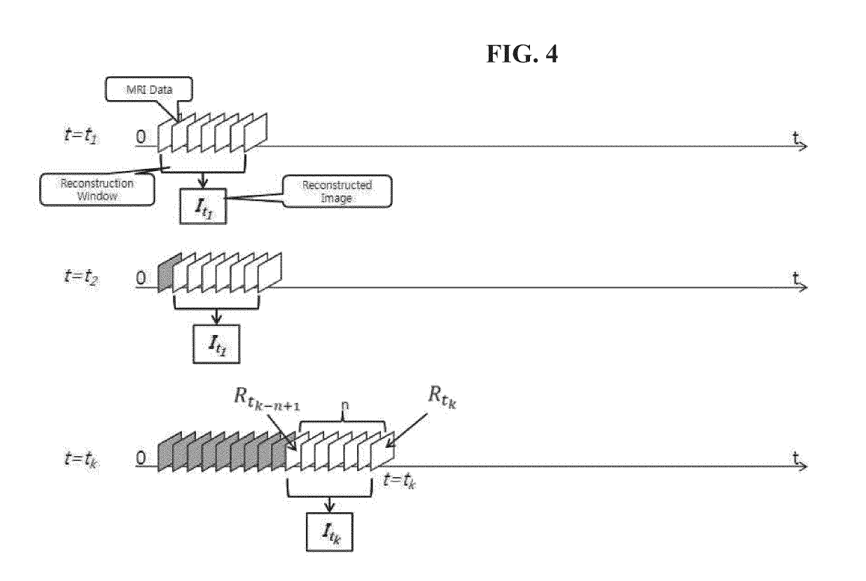

FIG. 4 is a drawing for the purpose of explaining an example of a tomographic correction unit producing corrected tomographic image in real time, according to an embodiment of the present invention.

Referring to FIG. 4, when the window size range is “n”, the number of tomographic image data obtained from t₀ to t₁ may be n. Accordingly, the time point at which the earliest corrected tomographic image is produced is t₁, and multiple ultrasonic images and one or more tomographic image data included in the window size range with the next time point as reference are used to produce corrected tomographic image.

In other words, in order to produce corrected tomographic image in real time, the ultrasonic imaging unit 210 and the tomographic imaging unit 220 obtain ultrasonic images and tomographic image data in real time, and the tomographic correction unit 230 uses multiple ultrasonic images and one or more tomographic image data included within the window size range with the present time as reference to reconstruct tomographic image, thus producing corrected tomographic image.

Here, the corrected tomographic image may be expressed as in Formula 2 below.

$${I}_{{t}_{k}} = {\sum }_{j = k - n + 1}^{k} f ( {R}_{{t}_{j}} , {mv}_{j , k} ) \qquad [ Formula 2 ]$$

Here, Iₜ_{ₖ} stands for the corrected tomographic image produced at time t=tₖ, Rₜ_{ⱼ} stands for tomographic image data obtained at time t=tₖ, n stands for window size range, mv_{j,k} stands for a motion vector indicating motion from Rₜ_{ⱼ} to Rₜ_{ₖ}, and f stands for a function applying correction information to tomographic image data.

In this manner, according to an embodiment of the present invention, when the tomographic correction unit 230 produces corrected tomographic image by using multiple ultrasonic images and one or more tomographic image data existing within the window size range with the present time as reference, tomographic image data may be corrected with the use of motion information of existing ultrasonic images produced and motion information generated for ultrasonic images newly added when the reference point changes, corrected tomographic image may be produced without a break in real time.

In other words, when producing corrected tomographic image at the present time, the information that was used at the time of producing corrected tomographic image at the previous time point is used as is, and only the handling process for ultrasonic images and tomographic image data newly added in the window size range at the present time point is added, corrected tomographic image may be produced without a break in real time.

Meanwhile, according to an embodiment of the present invention, the tomographic correction unit 230, after selecting each of the multiple ultrasonic images as a reference ultrasonic image, performs the extraction of the motion information, correction of the one or more tomographic image data, and production of the corrected tomographic image, described above in FIG. 3, as many times as the multiple number, thus producing multiple corrected tomographic images, and structures the multiple corrected tomographic images into moving images.

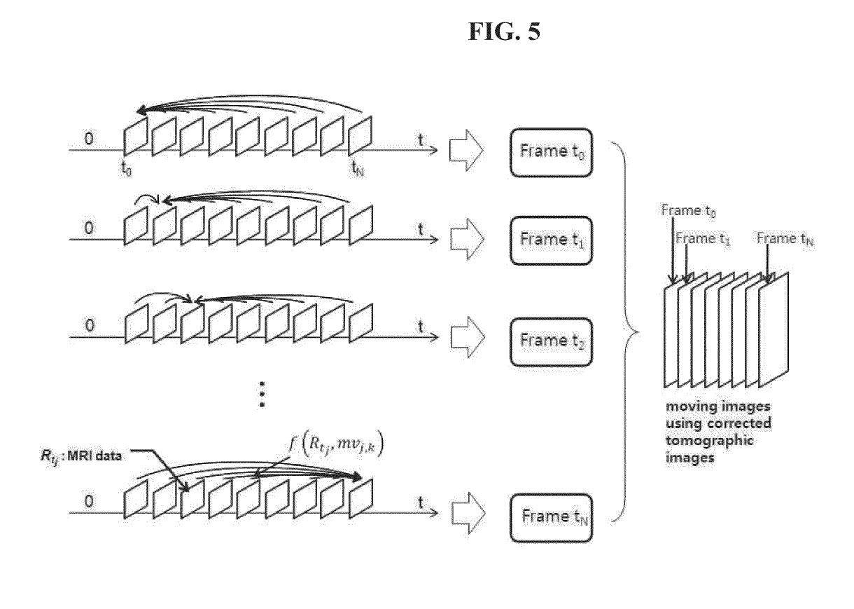

In other words, as illustrated in FIG. 5, the tomographic correction unit 230 produces N+1 number of corrected tomographic images (from Frame t₀ to Frame t_{N}) with each of the ultrasonic images obtained from Frame t₀ to Frame t_{N} as a reference ultrasonic image, and indicates the N+1 number of corrected tomographic images according to the flow of time, thus indicating the tomographic images corrected as moving images.

At the same time, according to an embodiment of the present invention, the tomographic image producing apparatus 200 may comprise medical equipment that uses tomographic image together with a treatment/biopsy unit (not pictured) that performs treatment or biopsy for a subject area by the use of the corrected tomographic image produced

In other words, when corrected tomographic image is produced in real time, verification of the position of a subject and its tracing becomes possible, and therefore, treatment/biopsy of the subject in real time may be performed by means of a treatment/biopsy unit.

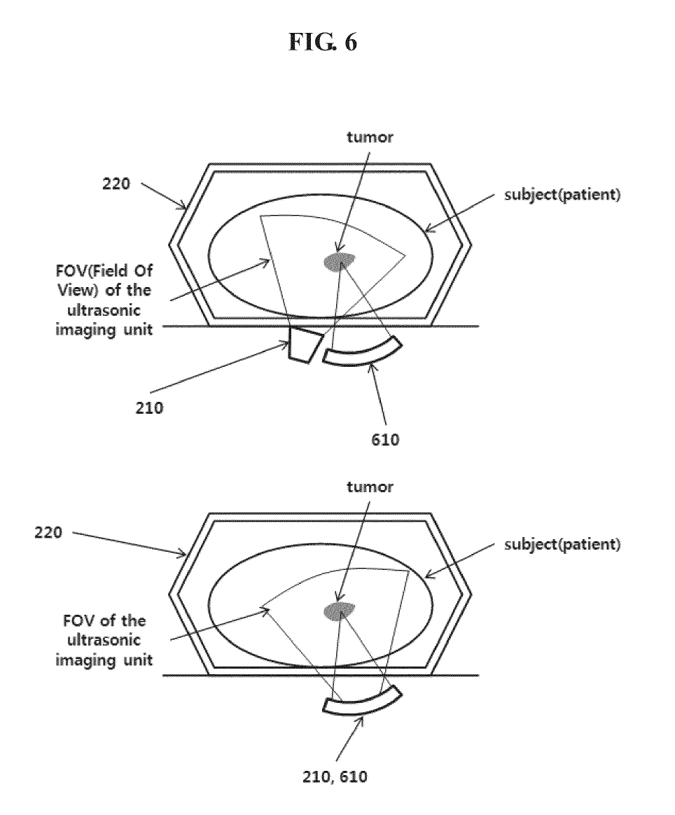

FIG. 6 is a drawing illustrating an example of medical equipment using tomographic image according to an embodiment of the present invention.

FIG. 6(a) is a drawing illustrating an example of medical equipment that performs treatment of a subject by means of a treatment/biopsy unit 610. Referring to FIG. 6(a), the tomographic correction unit 230 produces the corrected tomographic image in real time by correcting tomographic image data obtained at the tomographic imaging unit 220 using ultrasonic images obtained through the ultrasonic imaging unit 210 and the treatment/biopsy unit 610 is able to perform treatment with the use of tomographic image produced in real time. In this case, for performing treatment and biopsy more accurately, the tomographic correction unit 230 can track the treatment/biopsy area (as an example, tumor) by producing the corrected tomographic image in real time.

And FIG. 6(b) is a drawing illustrating an example of medical equipment with treatment/biopsy unit 610 and ultrasonic imaging unit 210 implemented as one.

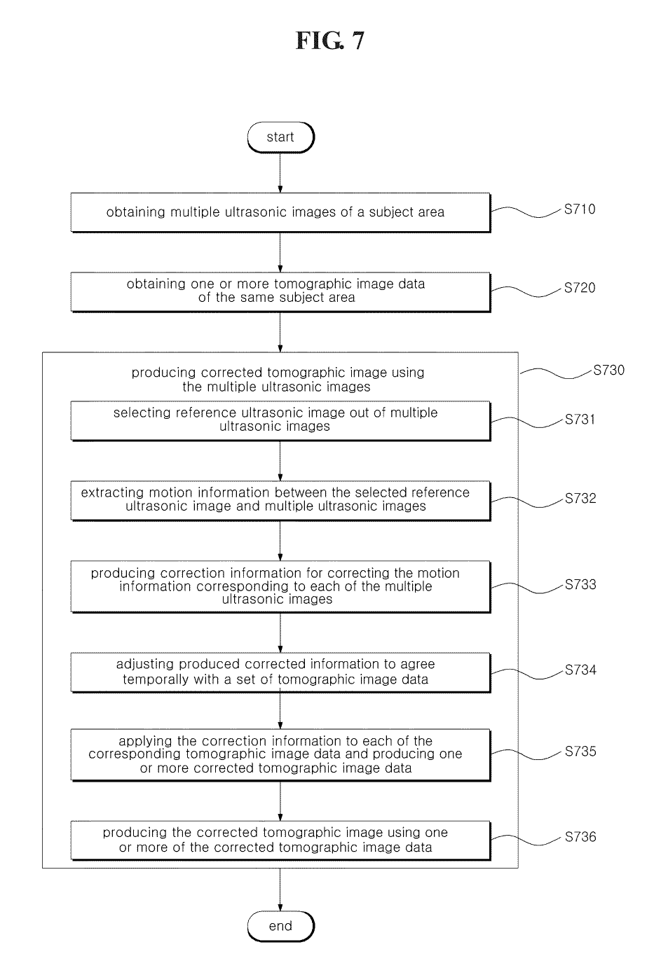

FIG. 7 is a flow chart illustrating the overall flow of the method of producing tomographic image according to an embodiment of the present invention.

The tomographic image production method described in FIG. 7 may be performed by the tomographic imaging apparatus 200 described in FIG. 2 above.

Below, operational process is described according to each step.

First, in operation S710, multiple ultrasonic images of a subject area are obtained, and in operation S720, one or more tomographic image data of the same subject area are obtained. Here, S710 and S720 should preferably be performed simultaneously.

Next, in operation S730, corrected tomographic image of a tomographic image, which can be produced from the one or more tomographic image data are produced with the use of the multiple ultrasonic images.

To give a more detailed explanation regarding this, a reference ultrasonic image is selected out of multiple ultrasonic images in operation S731, and motion information between the selected reference ultrasonic image and multiple ultrasonic images is extracted in operation S732.

Next, correction information for compensating the motion information corresponding to each of the multiple ultrasonic images is produced in operation S733, and the produced corrected information is adjusted to match temporally with a set of tomographic image data in operation S734.

Subsequently, the correction information is applied to each of the corresponding tomographic image data and one or more corrected tomographic image data are produced in operation S735, and one or more of the corrected tomographic image data are used to produce corrected tomographic image in operation S736.

While the above description has pointed out novel features of the invention as applied to particular embodiments, the embodiments are for illustrative purposes only and do not limit the invention, and those skilled in the art will understand that various omissions, substitutions, and changes in the form and details of the device or process illustrated may be made without departing from the scope of the invention. Therefore, the scope of the invention is defined by the appended claims rather than by the foregoing description, and all variations coming within the meaning and range of equivalency of the claims are embraced within their scope.

1. An apparatus for producing tomographic image, the apparatus comprising:
an ultrasonic imaging unit configured to obtain multiple ultrasonic images of a subject;
a tomographic imaging unit configured to obtain one or more tomographic image data of the subject; and
a tomographic image correction unit configured to produce corrected tomographic image of a tomographic image which can be produced from the one or more tomographic image data by using the multiple ultrasonic images.

2. The apparatus for producing tomographic image according to claim 1, wherein the tomographic correction unit selects a reference ultrasonic image out of the multiple ultrasonic images, extracts motion information between the reference ultrasonic image and the multiple ultrasonic images, corrects the one or more tomographic image data using the motion information, and produces the corrected tomographic image using the corrected one or more tomographic image data.

3. The apparatus for producing tomographic image according to claim 2, wherein the tomographic correction unit produces correction information for compensating the motion information corresponding to each of the multiple ultrasonic images, and adjusts the correcting information to match temporally with the one or more tomographic image data, producing the corrected one or more tomographic image data by applying the correction information to each of the corresponding one or more tomographic image data.

4. The apparatus for producing tomographic image according to claim 2, wherein the tomographic correction unit, after selecting each of the multiple ultrasonic images as a reference ultrasonic image, performs extraction of the motion information, correction of one or more of the tomographic image data and production of the corrected tomographic image as many times as the multiple number, thus producing multiple corrected tomographic images, and composes the multiple corrected tomographic images as moving images.

5. The apparatus for producing tomographic image according to claim 1, wherein the ultrasonic imaging unit obtains more than two ultrasonic images of the subject successively,
the tomographic imaging unit obtains more than two tomographic image data of the subject successively,
the multiple ultrasonic images are ultrasonic images included within a pre-designated window size range with the present time as the reference point out of the more than two ultrasonic images, and one or more of the tomographic image data are tomographic image data included within the window size range with the present time as the reference point, out of the more than two tomographic image data.

6. The apparatus for producing tomographic image according to claim 5, wherein the window size range means a minimum number of the tomographic image data obtained successively for producing the corrected tomographic image.

7. The apparatus for producing tomographic image according to claim 5, wherein tomographic correction unit selects a reference ultrasonic image out of the multiple ultrasonic images, extracts motion information between the reference ultrasonic image and the multiple ultrasonic images, corrects the one or more tomographic image data using the motion information, and produces the corrected tomographic image using the corrected one or more tomographic image data,
and the reference ultrasonic image is the most recently obtained ultrasonic image out of the multiple ultrasonic images included within the window size range, from the present time.

8. The apparatus for producing tomographic image according to claim 5, wherein the tomographic correction unit selects a reference ultrasonic image out of the multiple ultrasonic images, extracts motion information between the reference ultrasonic image and the multiple ultrasonic images, produces correction information for compensating the motion information corresponding to each of the multiple ultrasonic images, and adjusts the correcting information to match temporally with the one or more tomographic image data, corrects the one or more tomographic image data by applying the correction information to each of the corresponding one or more tomographic image data, and produces the corrected tomographic image using one or more of the corrected tomographic image data,
the motion information and the correction information are expressed as motion vectors,
and the corrected tomographic image is expressed as in the formula below:
$${I}_{{t}_{k}} = {\sum }_{j = k - n + 1}^{k} f ( {R}_{{t}_{j}} , {mv}_{j , k} )$$
Here, Iₜ_{ₖ} stands for the corrected tomographic image produced at time t=tₖ, Rₜ_{ⱼ} stands for tomographic image data obtained at t=tₖ, n stands for the window size range, mv_{j,k} stands for the motion vector indicating motion from Rₜ_{ⱼ} to Rₜ_{ₖ}, and f stands for the function that applies the correction information to the tomographic image data.

9. The apparatus for producing tomographic image according to claim 1, wherein the ultrasonic imaging unit and the tomographic imaging unit operate in temporal synchronization.

10. The apparatus for producing tomographic image according to claim 1, wherein the tomographic imaging unit includes at least one of the following: magnetic resonance imaging (MRI) equipment, computed tomography (CT) equipment, positron emission tomography (PET) equipment, and single photon emission computed tomography (SPECT) equipment.

11. The apparatus for producing tomographic image according to claim 1, further comprising a display unit for displaying the corrected tomographic image.

12. The apparatus for producing tomographic image according to claim 1, wherein the ultrasonic image producing unit comprises
an ultrasonic probe configured to radiate ultrasounds; and
an ultrasonic probe support for supporting the ultrasonic probe and adjusting the position of the ultrasonic probe.

13. A method for producing tomographic image, the method comprising:
obtaining multiple ultrasonic images of a subject;
obtaining one or more tomographic image data of the subject; and
producing corrected tomographic image of a tomographic image which can be produced from the one or more tomographic image data by using the multiple ultrasonic images.
